(19) United States
(12) Patent Application Publication
(10) Pub. No.: US 2016/0287846 A1
(43) Pub. Date: Oct. 6, 2016

(54) Medical Tube Fixing Apparatus and Medical Tube Fixing Method Using the Same

(71) Applicant: ZIONMED CO.,LTD., Seoul (KR)

(72) Inventor: Suk-il Lee, Seoul (KR)

(73) Assignee: ZIONMED CO., LTD., Seoul (KR)

(21) Appl. No.: 15/034,926

(22) PCT Filed: Nov. 8, 2013
(86) PCT No.: PCT/KR2013/010099

§ 371 Date: May 6, 2016

(30) Foreign Application Priority Data
Nov. 7, 2013 (KR) 10-2013-0134840

(51) Int. Cl. A61M 27/00; A61B 17/068; A61B 17/04; A61B 17/00

(52) U.S. Cl. CPC A61M 27/00; A61B 17/00234; A61B 17/068; A61B 17/0469; A61M 2210/1003

(57) ABSTRACT

Disclosed is a medical tube fixing apparatus which can fix a medical tube and simultaneously suture an incised surgical site. The medical tube fixing apparatus includes a body for fixing and supporting the medical tube, and a fixing pin, fixed to the body, for being fixed to skin of a patient while suturing the skin of the patient. The body includes a first fixing member, a second fixing member fixed to and supported by one lateral end of the first fixing member, and a locking unit by which the first and second fixing members are fastened and fixed to face each other. The first and second fixing members are formed with a plurality of protrusions on an inner side thereof, respectively. The fixing pin, being fixed to both of the first and second fixing members, can be fixed to the skin of the patient with a skin stapler.

### CROSS-REFERENCE TO RELATED APPLICATION

This application is a U.S. National Stage Application of International application No. PCT/KR2013/010099, filed on Nov. 8, 2013 and published in Korean as WO2015/068870 on May 14, 2015. This application claims priority to and benefit of Korean Patent Application No. 10-2013-0134840, filed on Nov. 7, 2013 before the Korean Intellectual Property Office (KIPO), the disclosures of which are incorporated by reference in their entirety herein.

### BACKGROUND

1. Technical Field

The present invention relates to an apparatus for fixing a medical tube directly to the skin of a patient with a skin stapler and a method of fixing the medical tube using the same, and more particularly, to a medical tube fixing apparatus which can suture an open surgical site and at the same time fix the medical tube in a manner of simply pressurizing with the skin stapler, and a medical tube fixing method using the same.

2. Discussion of the Related Art

In general, body fluids such as blood, pus, or the like may stagnate within a body of a patient after surgery of the patient. In that case, medical personnel may use a drain device to quickly drain the body fluids generated from the organs of the patient to the outside for a quick recovery of the patient.

The drain device is equipped with a medical tube for discharging the body fluids to the outside of the patient's body. There are widely used medical tubes such as a catheter that is used for discharging bile, abscess or the like from the liver, stomach or the like to the outside of the human body, an uretera catheterl that is inserted into a bladder and discharges urine, and a drainage tube, of which one end portion is inserted into a human body and the remaining portion is exposed to the outside, for discharging the body fluids generated from the internal organs to the outside of the body, etc.

Meanwhile, recently the medical skin stapler has been widely used to seal the surgical site in a state that the medical tube is inserted into the body of the patient after surgery of the patient. The skin stapler is a tool to improve the conventional method of suturing the incised skin caused by a surgical operation or a cosmetic surgery with a suture thread and a needle, and means a device for suturing the incised skin using the staples. The staple used by the skin stapler, having a substantially ‘ [symbol] -shape’, is loaded in a cartridge of the skin stapler and its both ends are bended inwards to suture the incised skin when the staple is pushed out by the skin stapler. In a sealed state of the incised skin like this, to fix a part of the medical tube exposed to the outside to the patient's body, a conventional medical band or a method of directly binding it to the skin with a thread has been used.

However, the medical band is disadvantageous in that it requires frequent replacement because of to weakened adhesion due to its long-time use. It has also other problem that a space is made between the medical band and the medical tube whenever the patient moves and thus the medical tube is pushed out to the outside due to the weakened adhesion.

In addition, there may be some other problems. The medical tube may be pushed out or the organs may be damaged due to insufficient adhesion of the medical band. As the medical tube is pulled out, infection caused by leakage of the pleural fluid, etc., pulmonary emphysema due to air infiltration, etc. may occur.

Furthermore, the binding method with the thread is not so robust and thus there is a problem that the medical tube inserted into the patient's body is obliged to escape from the body to the outside due to movement of the medical tube.

In consideration of these problems, Korean Patent No. 10-1064844 has disclosed “AN APPARATUS FOR FIXING A MEDICAL TUBE”.

The apparatus for fixing the medical tube disclosed by Korean Patent No. 10-1064844 provides a fixing apparatus of the medical tube that is installed through the skin of the patient. The fixing apparatus includes a tape formed with a tube through-hole for penetration of a tube at the center thereof and formed with an adhesive layer to be attached to the skin of a patient on a bottom surface thereof; a tube guiding member including a tape fixing member of which top surface or bottom surface is fixed to the vicinity of the tube through-hole, and a cap insertion member formed in a cylindrical shape on the tape fixing member, wherein a cap mounting hole is formed through the center of the tape fixing member and the cap insertion member; a fixing cap, formed at the center with a guide hole through which the tube is inserted, including a supporting member formed in a plate type along the circumference of the guide hole, and a plurality of pressing pieces of which outer circumference surfaces are downwardly slanted to the center and which are formed on the bottom surface of the supporting member and spaced apart from each other along the circumference of the guide hole, wherein inner surfaces of the pressing pieces tightly hold the tube, and outer surfaces of the pressing pieces are inserted into the cap mounting hole of the tube guide member so that outer surfaces of the pressing pieces can be tightly fixed up by the inner wall of the cap insertion member.

However, the apparatus for fixing the medical tube according to the prior art has some disadvantages that its manufacturing cost is high because its elements such as the fixing cap, the tube guide member and the tape are manufactured separately, and it takes lots of time to fix the medical tube fixing apparatus that is fixed by the tape to the patient. There is also a problem that the apparatus for fixing the medical tube may not be firmly fixed and work loose so as to move in case of long-time use.

In addition, the fixing apparatus may cause skin irritation because the tape is directly attached to the skin. As the fixing apparatus is used in a manner that it is directly attached to a patient's body and thus is used as a disposable one, when it is required that the apparatus for fixing the medical tube should be replaced, the entire replacement is needed, which results in high costs.

### SUMMARY

The present disclosure is directed to providing a medical tube fixing apparatus that can lower its production costs as being easy to produce, which can reduce the health care costs of patients, and can allow the surgical practitioner's quick and easy installation of the medical tube in a manner of simply stapling with the skin stapler so that the surgical practitioner can suture a cut-opened surgical site and simultaneously fix the medical tube.

In addition, the present disclosure is directed to providing a medical tube fixing method by which contact dermatitis due to the adhesive having a large area can be prevented, and the medical tube fixing apparatus can be made in a significantly small size such that the patient has no inconvenience in his or her movement.

The various problems intended to be solved by the present disclosure are not limited to those mentioned above, and any other problems that are not mentioned above will be clearly understood by one of ordinary skill in the art from the following description.

A medical tube fixing apparatus according to an exemplary embodiment of the present disclosure may include: a body for fixing and supporting a medical tube; and a fixing pin, fixed to the body, for suturing skin of a patient while being fixed to the skin of a patient. The body may include a first fixing member formed with a plurality of protrusions on an inner side thereof; a second fixing member, fixed to and supported by one side of the first fixing member, and formed with a plurality of protrusions on an inner side thereof; and a locking unit by which the first and second fixing pieces are locked to face each other. The fixing pin, being fixed to both of the first and second fixing members, can be fixed to the skin of the patient by being stapled with a skin stapler.

The body may be tightly engaged with the medical tube by the locking unit when the body loaded in the skin stapler is pressurized by the skin stapler, and the fixing pin provided in the body may suture an incised site of the patient and at the same time be inserted into and fixed to the skin of the patient when the body is further pressurized by the skin stapler.

The body may be formed in a cylindrical shell shape of which inside is vacant, and an inner diameter of the body may be smaller than an outer diameter of the medical tube so that the body can tightly grip the medical tube.

The locking unit may comprise a first supporting plate formed with a through-hole, and a second supporting plate comprising a protrusion to be press-fitted into the through-hole. And, the first supporting plate may be fixed to the first fixing member and the second supporting plate may be fixed to the second fixing member.

The fixing pin may be fixed to both of the first supporting plate and the second supporting plate.

The body may include an inner layer made of rubber or silicon having a non-slip function.

In addition, a method of fixing a medical tube according to an exemplary embodiment of the present disclosure may include: inserting the medical tube into a patient's body to drain out blood or pus within a patient's body; inserting the medical tube into the medical tube fixing apparatus so as to make the medical tube be fixed to the medical tube fixing apparatus; making the medical tube fixing apparatus be in contact with skin of a patient using a staple ejected from a skin stapler; and fixing the medical tube fixing apparatus to the skin of the patient by pressurizing the medical tube fixing apparatus with the skin stapler.

The medical tube fixing apparatus may be fixed to an incised surgical site of the patient's body or a position of the patient's body spaced apart from the incised surgical site.

Specific details of other embodiments are included in the detailed description and drawings below.

The medical tube fixing apparatus according to the exemplary embodiment of the present disclosure is capable of lowering its production costs and reducing the health care costs of patients as being easy for production. Furthermore, with the medical tube fixing apparatus, a surgical practitioner can suture the incised surgical site and at the same time fix the medical tube in a manner of simple operation with the skin stapler, thereby having advantages of quickly completing installation of it and providing the surgical practitioner with convenience.

In addition, the medical tube fixing apparatus according to the exemplary embodiment of the present disclosure is capable of preventing contact dermatitis which may be caused by the adhesive having a large area, and removing the patient's inconvenience for movement as the medical tube fixing apparatus can be produced in a significantly small size.

It may be fully understood that various exemplary embodiments of the technical spirit of the present disclosure may provide a variety of effects that are not specifically mentioned here.

### BRIEF DESCRIPTION OF THE DRAWINGS

FIGS. 1 and 2 illustrate perspective views of a medical tube fixing apparatus according to an exemplary embodiment of the present disclosure.

FIG. 3 illustrates a cross-sectional view of the medical tube fixing apparatus according to the exemplary embodiment of the present disclosure.

FIG. 4 illustrates a state in which the medical tube fixing apparatus is used according to an exemplary embodiment of the present disclosure.

FIGS. 5 and 6 illustrate a state in which the medical tube fixing apparatus is used in accordance with an exemplary other embodiment of the present disclosure.

FIGS. 7 and 8 illustrate a medical tube fixing apparatus according to another exemplary embodiment of the present disclosure.

### DETAILED DESCRIPTION OF THE EMBODIMENTS

Advantages and features of the present inventive concept and methods of accomplishing the same will become apparent with reference to the embodiments that will be described below in detail, along with the accompanying drawings. The present inventive concept may, however, be embodied in many different forms and should not be construed as limited to the exemplary embodiments set forth herein. Rather, these exemplary embodiments are provided so that this disclosure will be thorough and complete, and will fully convey the scope of the present inventive concept to those skilled in the art. In the drawings, the sizes and relative sizes of layers and regions may be exaggerated for clarity.

It will be understood that, although the terms first, second, etc. may be used herein to describe various elements, these elements should not be limited by these terms. These terms are used just to distinguish one element from another. Thus, a first element may be termed a second element without departing from the teachings of the present inventive concept, and similarly the second element may be termed the first element.

The terminology such as the upper part, the lower part, the top surface, the top, the bottom, etc. will be used to distinguish between the relative positions of the components. For example, in case of naming the part above and the part below the middle of a drawing as the upper part and the lower part, in practice without departing from the scope of the present inventive concept the upper part may be referred to as the lower part, and the lower part may be referred to as the upper part.

The terminologies used herein are for the purpose of describing particular exemplary embodiments only and are not intended to be limiting of the present inventive concept. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms, including technical and scientific terms, used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

With reference to the accompanying drawings, preferred embodiments of the medical tube fixing apparatus according to the present inventive concept will be described in detail.

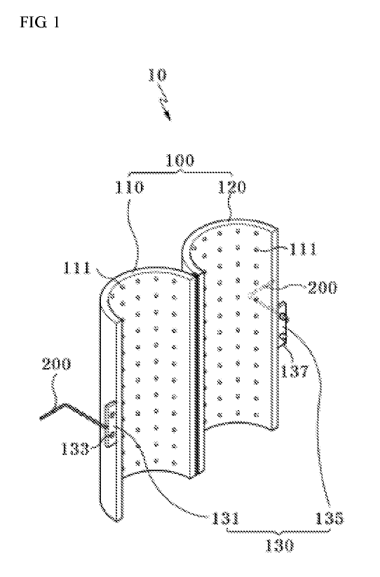

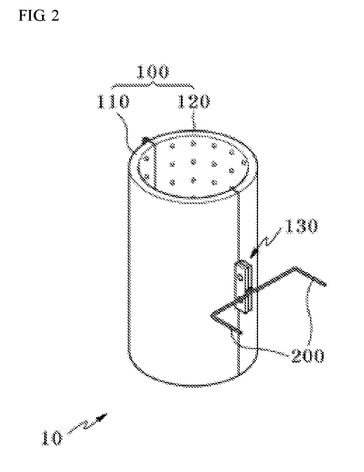

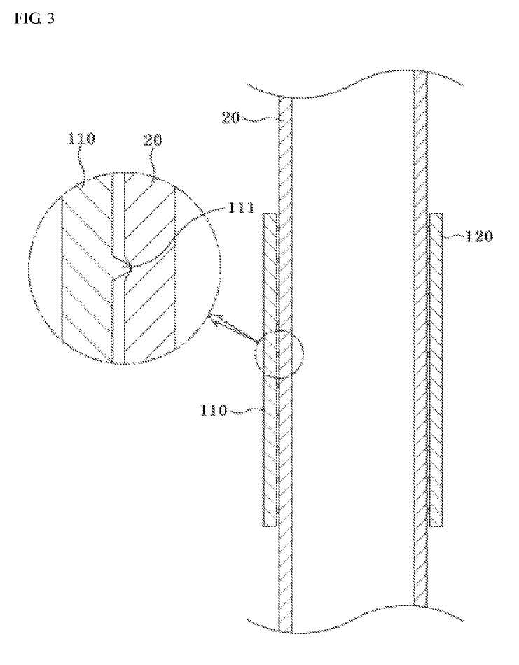

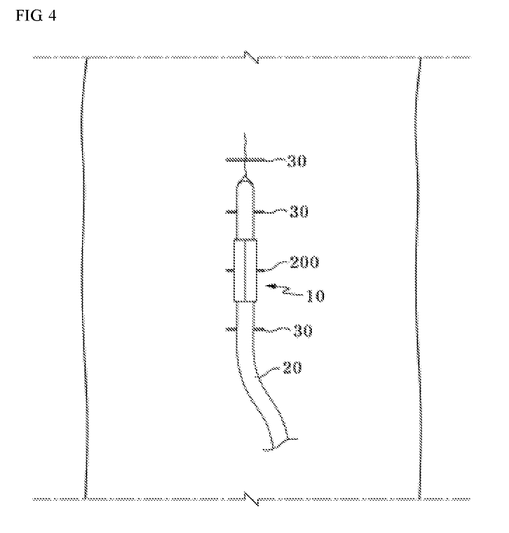

FIGS. 1 and 2 are perspective views of the medical tube fixing apparatus according to an exemplary embodiment of the present disclosure. FIG. 3 is a cross-sectional view of the medical tube fixing apparatus according to the exemplary embodiment of the present disclosure. FIG. 4 illustrates a state in which the medical tube fixing apparatus is used according to an exemplary embodiment of the present disclosure.

With reference to FIGS. 1 to 4, the medical tube fixing apparatus 10 according to the exemplary embodiment of the present disclosure is a device for fixing a medical tube 20 to drain out quickly body fluids such as blood, pus, or the like which may stagnate within a patient's body after surgery.

The medical tube fixing apparatus 10, which may be used to suture a surgical site of a patient and at the same time to fix firmly the medical tube 20 inserted into the patient's body, may include a body 100 and one or more fixing pins 200.

The body 100 may be provided so as to fix and support the medical tube 20. The body 100 is formed in a cylindrical shell shape of which inside is vacant, and its inner diameter may be smaller than the outer diameter of the medical tube 20. The inner side of the body 100 may be made of rubber or silicone to provide a non-slip function. The body 100 may include two semi-cylindrical members, where one is referred to as a first fixing member 110 and the other is referred to as a second fixing member 120, and these two semi-cylindrical members may be obtained by cutting a cylindrical shell into two half-pieces in its axial direction. A pair of first lateral ends of the two semi-cylindrical members are fixed to each other, and a pair of second lateral ends of them can be opened, and thus the two semi-cylindrical members can be pivoted around the first lateral ends. The body 100 may further include a locking unit 130.

The first fixing member 110 may be provided with a number of protrusions 111 that are spaced apart from each other by a predetermined interval on its inner side (wall) and have an emboss-effect.

The second fixing member 120 may be fixed to and supported by a lateral end of the first fixing member 110, and may be provided with a number of protrusions 111 that are spaced apart from each other by a predetermined interval on its inner side (wall) and have the emboss-effect.

That is, one lateral end (hereinafter, referred to as ‘a first end’) of the first fixing member 110 and one lateral end (hereinafter, referred to as ‘a first end’) of the second fixing member 120 may be fixedly connected to each other, whereas the other lateral end (hereinafter, referred to as ‘a second end’) of the first fixing member 110 and the other lateral end (hereinafter, referred to as ‘a second end’) of the second fixing member 120 may not be connected so as not to be opened, which allows either the first fixing member 110 or the second fixing member 120 to be pivoted around the other in a circumferential direction.

The locking unit 130 may be provided to lock the first fixing member 110 and the second fixing member 120 together. The locking unit 130 may include a first supporting plate 131 and a second supporting plate 135.

The first supporting plate 131 may be fixed to the second end of the first fixing member 110 and formed with one or more through-holes 133 through which the second supporting plate 135 can be secured to the first supporting plate 131.

The second supporting plate 135 may be fixed to the second end of the second fixing member 120 and may be provided with one or more protrusions 137 that can be press-fitted into the through-holes 133 and fixed to the first supporting plate 131.

The first supporting plate 131 and the second supporting plate 135 may be fastened and fixed to each other by being simply pressurized just one time with a skin stapler (not shown).

The locking unit 130 may have any other structure and/or element if it can allow the first fixing member 110 and the second fixing member 120 to be secured and fastened to each other. For example, the locking unit 130 may be formed with a male member and a female member both of which can be fastened to each other in the manner of simply pressurizing.

The fixing pin(s) 200 may be fixed to both of the first fixing member 110 and the second fixing member 120 respectively, and inserted into and fixed to the skin of the patient in the manner of simply pressurizing with the skin stapler (not shown). The fixing pins 200 may be fixed to the first supporting plate 131 and the second supporting plate 135, respectively. In other words, the fixing pins 200 may be fixed to the first fixing member 110 and the second fixing member 120, respectively, or the first supporting plate 131 and the second supporting plate 135, respectively, relaying on the structure in which the locking unit 130 is formed.

In the above, the medical tube fixing apparatus 10 according to the exemplary embodiment according to the present disclosure is described with respect to an example of suturing the surgical site of the patient and at the same time firmly fixing the medical tube 20 inserted into the patient's body as shown in FIG. 4, but it should be noted that the medical tube fixing apparatus 10 according to the present disclosure is not limited to the example and can be used in variously modified forms by one of ordinary skill in the art.

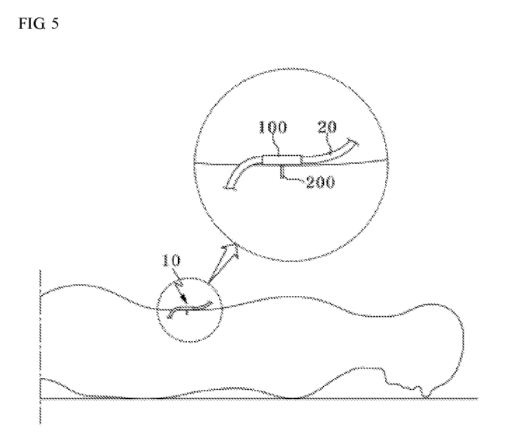

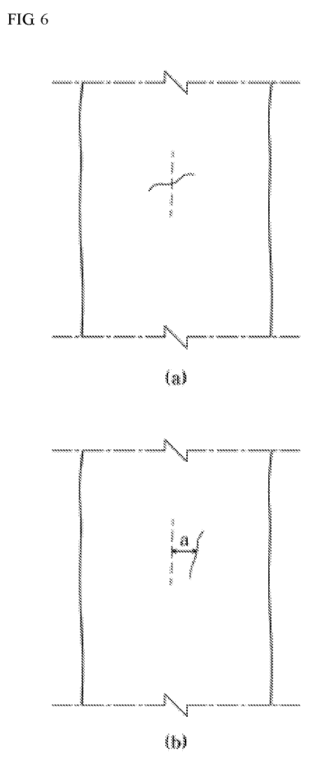

With reference to FIGS. 5 and 6, the medical tube fixing apparatus 10 according to the present disclosure can be fixed after the surgical site of a patient is sutured and another site on the skin around the surgical site is incised. For example, as shown in FIG. 6(a) the surgical site is sutured in accordance with the type of surgery, and as shown in FIG. 6(b) the body fluids such as blood, pus, or the like which may stagnate within the patient's body can be drained out quickly through the incised another site spaced apart from the sutured surgical site by a predetermined distance a. In this case, as shown in FIG. 5 after the surgical site is sutured and the medical tube 20 is firmly gripped by the body 100, the medical tube 20 is inserted into the another site around the surgical site and the fixing pins 200 can be inserted into and fixed to the patient's skin in the manner of simply pressurizing with the skin stapler.

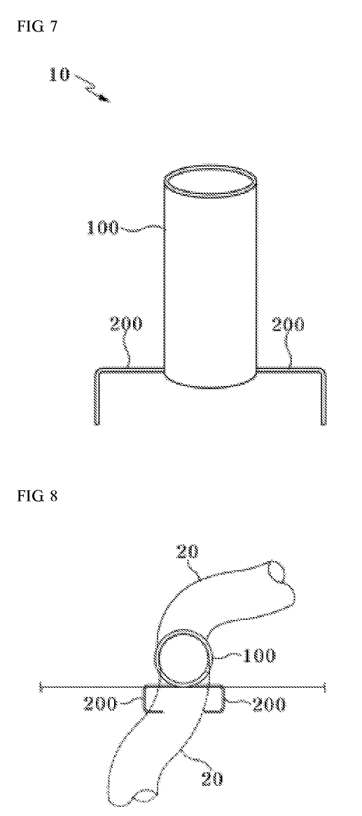

Also, various modifications of the medical tube fixing apparatus 10 according to the present disclosure can be made as shown in FIGS. 7 and 8. For example, as shown in FIG. 7 the fixing pins 200 may be provided at a position of the body 100, and end portions of the fixing pins 200 may be inserted into the patient's skin such that they can be bended.

Hereinafter, with reference to FIGS. 1 to 4 described in more detail is a fixing method of suturing the surgical site of the patient with the medical tube fixing apparatus 10 loaded in the skin stapler (not shown).

Here, the skin stapler may be the one that is suitably designed to secure the fixing pins 200 to the skin of the patient, and is not shown in the drawings for convenience of description.

First, in a state that staples 30 and the medical tube fixing apparatus 10 are received in a cartridge equipped in the skin stapler, the medical tube 20 is inserted into the patient's body to drain out the blood, pus, or the like collected within the surgical site.

In the state that the medical tube is inserted into the patient's body, the surgical practitioner sutures the surgical site with the skin stapler by pressurizing so as to eject the staples 30 and to seal the incised surgical site. In FIGS. 1 to 4, it is described as an example to suture the incised surgical site of the patient in the state that the medical tube 20 is inserted into the incised surgical site, but the present disclosure is not limited to this example. One of ordinary skill in the art may insert the medical tube 20 into another site spaced apart from the incised surgical site of the patient as illustrated in FIGS. 5 and 6.

Next, in the state that the medical tube 20 is in a close contact with the skin of the patient, if the skin stapler is primarily pressurized, the skin stapler works so as to make the medical tube fixing apparatus 10 firmly be coupled with the medical tube 20 and be located the outside of the patient's body.

Then, the skin stapler is pressurized secondly in order to suture the incised site and at the same time fix the medical tube fixing apparatus 10 to the patient's skin.

As such, when the secondary pressuring is applied to the skin stapler, the fixing pins 200 equipped in the medical tube fixing apparatus 10 are inserted into and bended so as to be fixed to the skin of the patient. Hereby the surgery may be ended up.

The foregoing is illustrative of example embodiments and is not to be construed as limiting thereof. Although a few example embodiments have been described, those skilled in the art will readily appreciate that many modifications are possible in the example embodiments without materially departing from the novel teachings and advantages of the present inventive concept. Accordingly, all such modifications are intended to be included within the scope of the present inventive concept as defined in the claims. Therefore, it is to be understood that the foregoing is illustrative of various example embodiments and is not to be construed as limited to the specific example embodiments disclosed, and that modifications to the disclosed example embodiments, as well as other example embodiments, are intended to be included within the scope of the appended claims.

What is claimed is:

1. A medical tube fixing apparatus, comprising:
a body fixing and supporting a medical tube; and
a fixing pin, fixed to the body, for suturing and being fixed to skin of a patient,
wherein the body comprises a first fixing member formed with a plurality of protrusions on an inner side thereof; a second fixing member, fixed to and supported by an side of the first fixing member, and formed with a plurality of protrusions on an inner side thereof; and a locking unit by which the first and second fixing members are locked to face each other, and
wherein the fixing pin, being fixed to both of the first and second fixing members respectively, for being fixed to the skin of the patient being stapled with a skin stapler.

2. The medical tube fixing apparatus according to claim 1, wherein the body is tightly engaged with the medical tube by the locking unit when the body loaded in the skin stapler is pressurized, and the fixing pin provided in the body sutures an incised site of the patient and at the same time is inserted into and fixed to the skin of the patient when the body is further pressurized by the skin stapler.

3. The medical tube fixing apparatus according to claim 1, wherein the body is formed in a cylindrical shell shape of which inside is vacant, and an inner diameter of the body is smaller than an outer diameter of the medical tube so that the body can tightly grip the medical tube.

4. The medical tube fixing apparatus according to claim 1, wherein the locking unit comprises a first supporting plate formed with a through-hole; and a second supporting plate comprising a protrusion to be press-fitted into the through-hole, wherein the first supporting plate is fixed to the first fixing member and the second supporting plate is fixed to the second fixing member.

5. The medical tube fixing apparatus according to claim 1, wherein the fixing pin is fixed to the first supporting plate and the second supporting plate.

6. The medical tube fixing apparatus according to claim 1, wherein the body comprises an inner layer made of rubber or silicon having a non-slip function.

7. A method of fixing a medical tube, comprising:
inserting the medical tube into a patient's body to drain out blood or pus within a patient's body;
inserting the medical tube into the medical tube fixing apparatus so as to make the medical tube be fixed to the medical tube fixing apparatus;
making the medical tube fixing apparatus be in contact with skin of a patient using a staple ejected from a skin stapler; and
fixing the medical tube fixing apparatus to the skin of the patient by pressurizing the medical tube fixing apparatus with the skin stapler.

8. The method of fixing the medical tube according to claim 7, wherein the medical tube fixing apparatus is fixed to an incised surgical site of the patient's body or a position of the patient's body spaced apart from the incised surgical site.
